(19) United States
(12) Patent Application Publication
(10) Pub. No.: US 2005/0187529 A1
(43) Pub. Date: Aug. 25, 2005

(54) Waste collection unit

(76) Inventors: Stephen J. Reasoner, Kalamazoo, MI (US); Timothy William Austin, Scotts, MI (US); Glen Rocque, Portage, MI (US); Karen Staley, Kalamazoo, MI (US); Jon Hietbrink, Grand Blanc, MI (US); Mark Wasserman, Delton, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/060,665

(22) Filed: Feb. 17, 2005

Related U.S. Application Data
(60) Provisional application No. 60/545,144, filed on Feb. 17, 2004.

(51) Int. Cl. A61M 1/00

(52) U.S. Cl. 604/317

(57) ABSTRACT

A waste collection unit is provided for collecting medical waste such as bodily fluids and materials from a patient, and disposing of the medical waste at a docking station. The waste collection unit comprises a canister for containing the medical waste and a carrier with wheels for transporting the waste collection unit between use areas, such as operating rooms, and the docking station. A vacuum housing with a plurality of vacuum ports is provided for connecting to an external vacuum source to draw a vacuum inside of the canister and collect the medical waste from the patient. The waste collection unit also includes a sprinkler system for cleaning the canister after disposing of the collected medical waste. The sprinkler system includes a sprinkler having a helical configuration in a cap of the canister for generating a cone-shaped spray pattern and a spray nozzle in a base of the canister.

### RELATED APPLICATIONS

This application claims the benefit of U.S. provisional patent application Ser. No. 60/545,144, filed Feb. 17, 2004, the advantages and disclosure of which are herein incorporated by reference.

### FIELD OF THE INVENTION

The present invention relates to a waste collection unit and more particularly to a waste collection unit that is part of a waste disposal system that employs the waste collection unit for collecting medical waste and a docking station for disposing of the medical waste from the waste collection unit.

### BACKGROUND OF THE INVENTION

Waste collection units are well known for use in surgical environments to collect medical waste such as bodily fluids and materials during a surgical procedure. Examples of waste collection units can be found in U.S. Pat. Nos. 5,997,733; 6,180,000; and 6,222,283. For instance, U.S. Pat. No. 5,997,733 discloses a waste liquid and smoke disposal system which combines the functions of a smoke extraction system and a waste collection unit, typically in, but not limited to, a surgical environment. The smoke extraction system and the waste collection unit are connected to supply the medical waste collected thereby to a waste treatment (e.g. decontamination and/or sterilization) and disposal system. In such systems, the waste collection unit can be provided as a cart-mounted apparatus to provide mobility. The waste collection unit can then dock to known docking stations to dispose of the medical waste collected by the unit. As a result, surgical teams can quickly, easily, and efficiently maintain the integrity of a surgical site with a minimum of operating components.

In these types of systems, once the medical waste is collected, the waste collection unit can then be wheeled to a docking station to be emptied and cleaned. The waste collection unit can be connected to the docking station without the need for any manual connections. This is greatly beneficial by reducing, if not eliminating, inadvertent contact with the contained medical waste. Although these types of waste collection units have been very successful, there is a need for increased benefits in a more economical unit. One benefit desired is the need to improve cleaning of the waste collection unit. The cleaning cycle typically employed to remove any residual medical waste in the waste collection unit during disposal is effective, but there is a need for an improved cleaning cycle that serves to more efficiently clean the waste collection unit. Also, prior waste collection units include an onboard vacuum system to draw the medical waste into a canister of the waste collection unit. To reduce the cost of these units, there is a need to eliminate the use of onboard vacuum systems, while still allowing the unit to be used and to be emptied without manual connections. Lastly, there is a need to improve the ease of docking the waste collection unit with the docking station.

### BRIEF SUMMARY OF THE INVENTION

The present invention provides a waste collection unit for collecting medical waste and disposing of the medical waste at a docking station. The waste collection unit comprises a canister having an inlet for receiving the medical waste and an outlet for discharging the medical waste at the docking station. The canister also includes a cap and a base with a wall extending between the cap and the base. A sprinkler is supported by the cap for directing a cleaning agent against the wall of the canister during a cleaning cycle after disposing of the medical waste at the docking station. The sprinkler includes a helical configuration for providing a cone-shaped spray pattern of the cleaning agent against the wall of the canister. This spray pattern has the advantage of effectively covering an interior surface of the wall of the canister with the cleaning agent, thus allowing the cleaning agent to work along a large portion of this surface to clean the canister.

The present invention also provides the waste collection unit with a control unit programmed for supplying the cleaning agent at a high concentration into the canister through a sprinkler system in a plurality of repeated injections during the cleaning cycle with pauses between the injections. By using high concentrations of cleaning agent applied to the canister in repeated injections, the canister can be cleaned in stages that can be controlled to efficiently clean the canister without wasting the cleaning agent.

The present invention also provides the canister with a vacuum housing having first and second vacuum ports in communication with the canister for selectively connecting to an external vacuum source to draw a vacuum within the canister thereby drawing the medical waste into the canister through the inlet. A check valve is disposed within the first vacuum port for preventing the drawing of air in through the second vacuum port while the first vacuum port is connected to the external vacuum source and the second vacuum port is disconnected from the external vacuum source. The vacuum ports allow the waste collection unit to utilize the external vacuum source provided in a use area, e.g., operating room, doctor's office, etc., thereby eliminating any onboard vacuum systems and reducing the cost of the waste collection unit, while still allowing the waste collection unit to be used and to be emptied without manual connections.

The present invention also provides a carrier supporting the canister. The carrier includes a plurality of wheels for providing mobility to the waste collection unit between the use areas and the docking station. A strike plate is floatably supported by the carrier to better align the waste collection unit with the docking station. By allowing the strike plate to float, docking the waste collection unit with the docking station does not require the precision currently needed with known units.

### BRIEF DESCRIPTION OF THE SEVERAL VIEWS OF THE DRAWINGS

Advantages of the present invention will be readily appreciated as the same becomes better understood by reference to the following detailed description when considered in connection with the accompanying drawings wherein:

FIG. 1 is a perspective view of a waste disposal system employing a waste collection unit of the present invention;

FIG. 2 is a perspective view of the waste collection unit showing a canister of the waste collection unit;

FIG. 3 is a perspective view of a cap of the canister;

FIGS. 4A and 4B are cross-sectional views of a vacuum housing of the waste collection unit;

FIG. 5A is an exploded view of a docking system of the waste collection unit;

FIGS. 5B and 5C are cross-sectional views of the docking system of FIG. 6A;

FIG. 6 is a schematic view of the waste disposal system illustrating the plumbing and electrical connections between the waste collection unit 12 and the docking station 14;

FIG. 7 is a cross-sectional view of the canister illustrating a spray pattern of a sprinkler system of the present invention;

FIG. 8 is a perspective view of a base of the canister without a float;

FIG. 9 is a perspective view of the base of the canister with the float; and

FIG. 10 is an exploded view of a battery compartment of the waste collection unit.

### DETAILED DESCRIPTION OF THE INVENTION

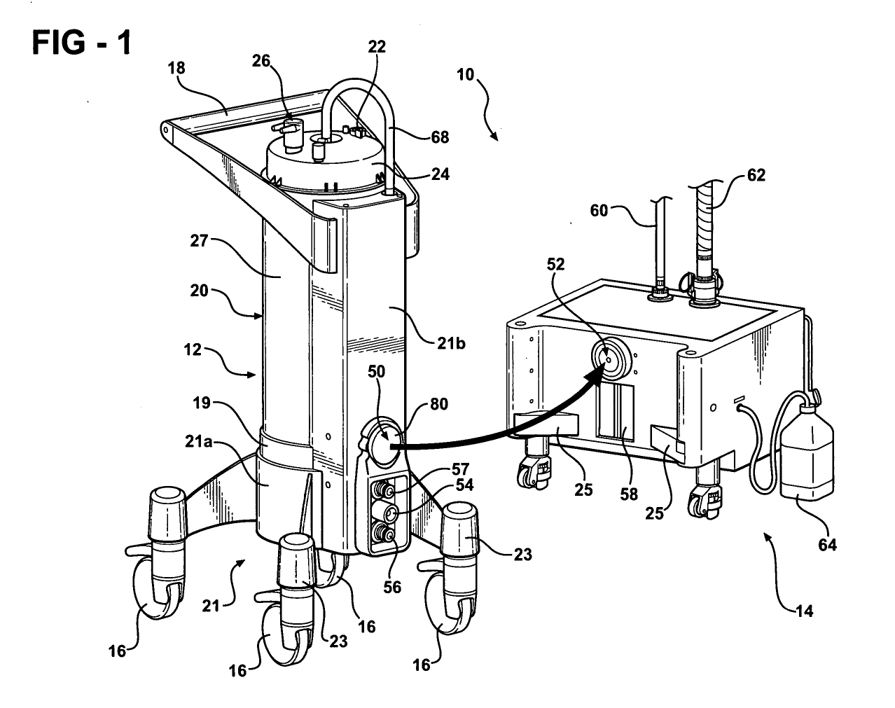

Referring to the Figures, wherein like numerals indicate like or corresponding parts throughout the several views, a waste disposal system for collecting and disposing of medical waste, e.g., bodily fluids and materials, is shown generally at 10. Referring to FIG. 1, the system 10 comprises a portable waste collection unit 12 for collecting the medical waste and a docking station 14 for disposing of the medical waste. The docking station 14 also provides a cleaning system for cleaning the waste collection unit 12 after disposing of the medical waste. The docking station 14 is currently sold as part of the NEPTUNE™ Waste Management System, commercially available from Stryker Instruments of Kalamazoo, Mich., the current assignee.

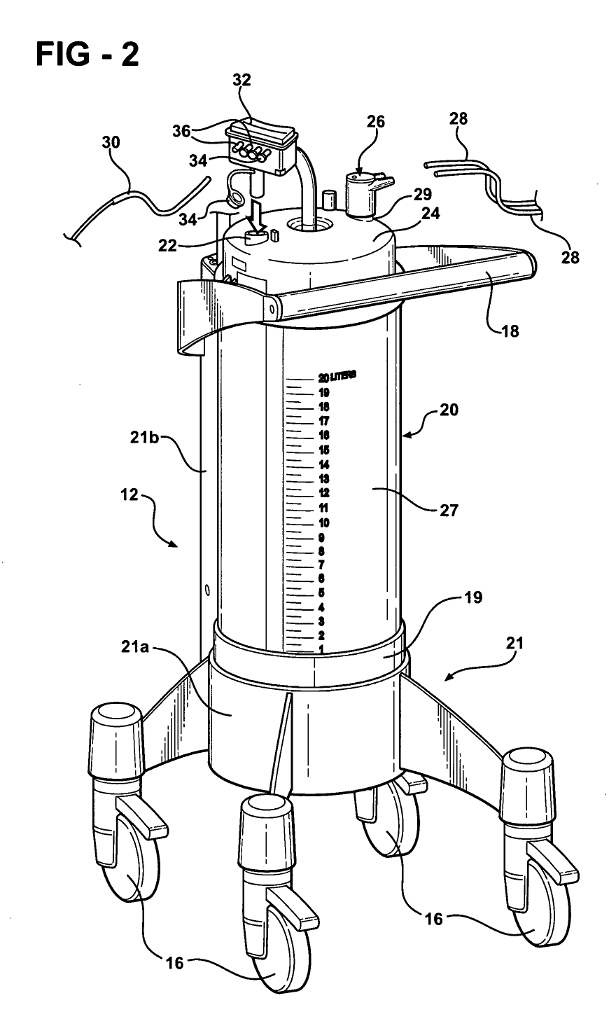

Referring to FIG. 2, the waste collection unit 10 has a collection canister 20 for containing the medical waste during use. The canister 20 includes a cap 24 and a base 19 with at least one wall 27 extending between the cap 24 and the base 19. The canister 20 preferably has a cylindrical shape. However, the canister may assume any shape that is suitable for containing medical waste. A carrier 21 supports the canister 20. More specifically, the carrier 21 includes a base 21a and support tower 21b to support the canister 20. The carrier 21 also includes a plurality of caster wheels 16 and a handle 18 to facilitate movement of the waste collection unit 12 between a use area, such as an operating room, and the docking station 14.

An inlet 22 is provided in the cap 24 of the canister 20 to receive medical waste from, for example, a patient having surgery in an operating room. A disposable manifold and filter system 32 is also shown at the inlet 22. The manifold and filter system 32 is used to facilitate the collection of the medical waste into the canister 20. The manifold and filter system 32 allows for various tubes 30 to be connected to inlet ports 36. The tubes 30 are connected at the other end to various waste collection tools (not shown). In this embodiment four inlet ports 36 are provided. Caps 34 can be used to close off unused inlet ports 36. The manifold and filter system 32 includes at least one filter (not shown) to remove solid or semi-solid material such as bone chips, flesh, blood clots or the like from the medical waste generated by the surgical procedure or operation. An example of such a manifold and filter system 32 is described in U.S. Pat. No. 6,331,246 to Beckham et al., herein incorporated by reference.

The medical waste is drawn into the canister 20 through the manifold and filter system 32 and the inlet 22 by a vacuum, which is applied through a vacuum housing 26. An external vacuum source is connected to the vacuum housing 26, such as the vacuum available in for example the operating room, doctor's office, etc. The vacuum source is connected to the waste collection unit 12 through tubes 28. There are two tubes 28 shown, but one would work and more than two would work as well depending upon the amount of vacuum available and needed.

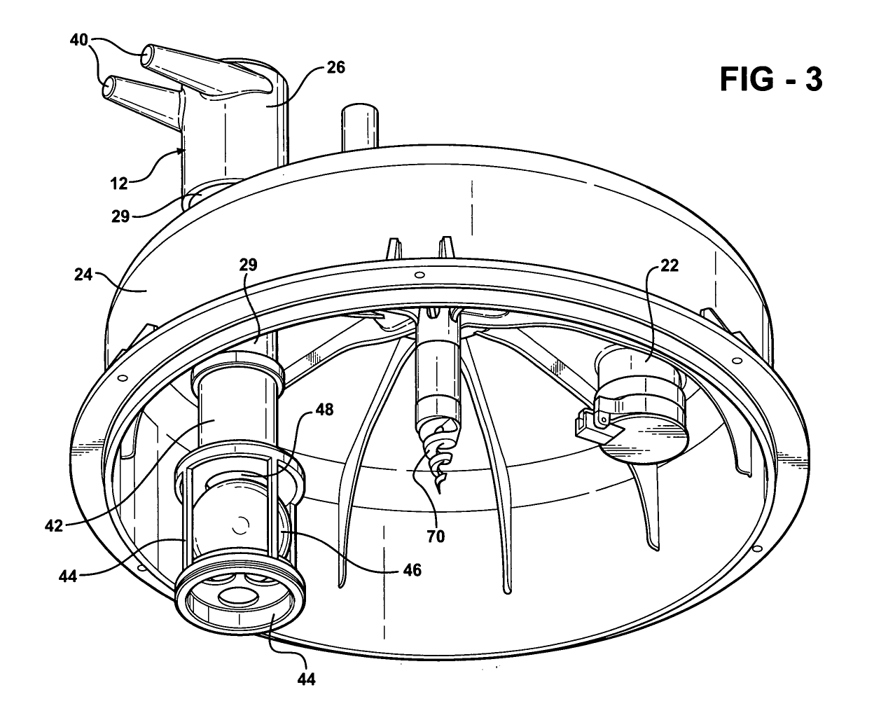

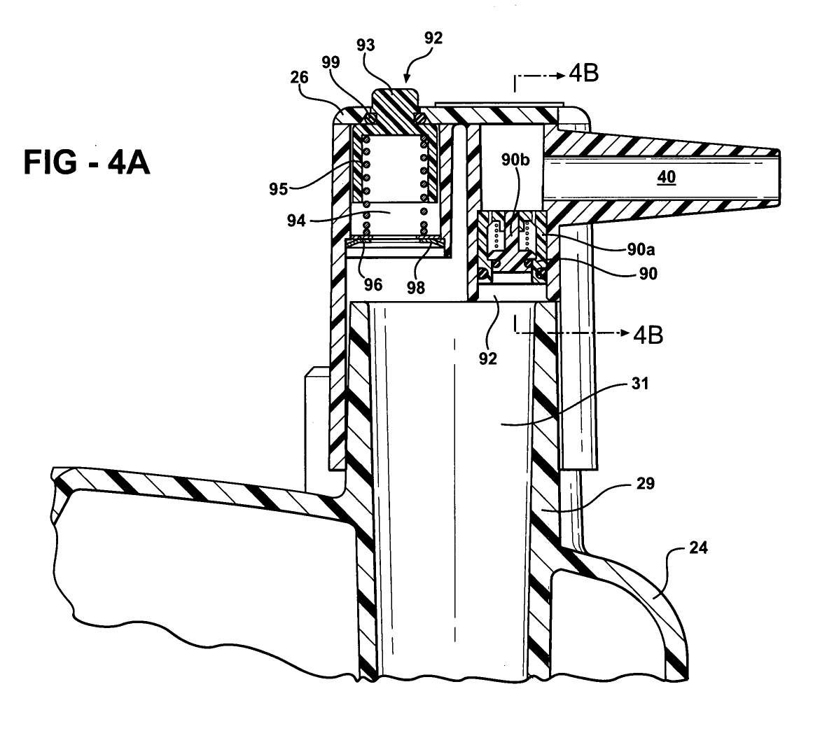

Referring to FIG. 3, the vacuum housing 26 is shown in greater detail. The vacuum housing 26 is illustrated with first and second vacuum ports 40 to which the collection tubes 28 can selectively be attached to draw a vacuum within the canister 20 thereby drawing the medical waste into the canister 20 through the inlet 22. The present invention is not limited to the use of two vacuum ports 40. Any number of vacuum ports 40 could be used. The cap 24 includes an integrally formed port member 29 that defines a port 31 (shown in FIGS. 4A-4B) through the cap 24. The vacuum housing 26 is mounted about the port 31 at a first end of the port member 29 outside of the canister 20 to provide communication between the vacuum ports 40 and the canister 20 and allow the vacuum to be drawn in the canister 20. The vacuum housing 26 is preferably formed of plastic and may be fixed to the port member 29 by any conventional method including adhesive, press fit, and the like.

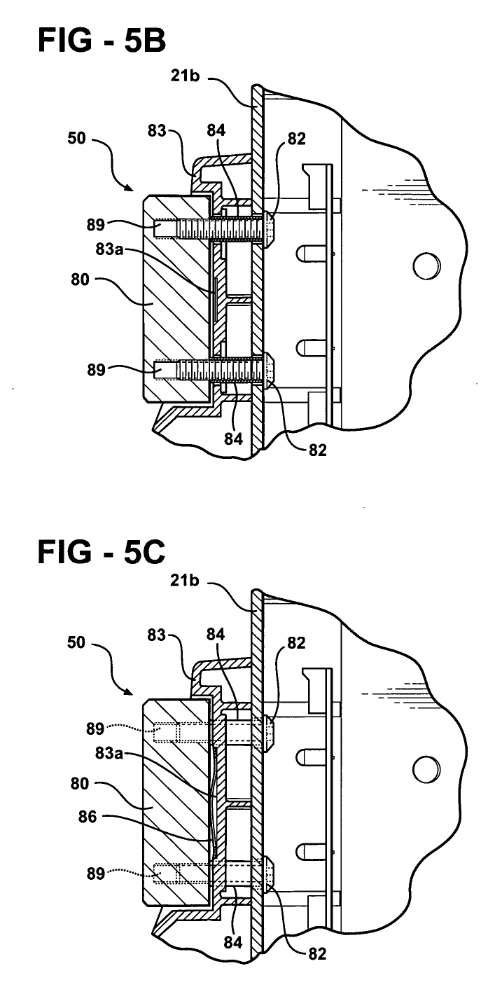

The port member 29 extends to a second end inside of the canister 20. A cage 44 is fixed to the second end of the port member 29. More specifically, the cage 44 includes a hollow extension 42 that is fixed to the second end of the port member 29 such as by adhesive. An opening 48 is defined in the extension 42. The opening 48 is in fluid communication with the port 31 such that the vacuum drawn through the vacuum housing 26 continues through the port 31 and the opening 48 into the canister 20. The cage 44 traps a float 46, which in the disclosed embodiment is a check ball, but could take other shapes or forms. The float 46 is heavy enough so that the vacuum being drawn in the canister 20 does not move the float 46. The float 46 only moves when the fluid within the canister 20 reaches the float 46. In this way, as the fluid level in the canister 20 is raised to the float 46, the float 46 raises. In the event the fluid level in the canister 20 continues to rise, the float 46 closes the opening 48 in the extension 42, thereby closing the communication between the vacuum ports 40 and the canister 20 when the medical waste is at a predetermined level. This prevents fluid in the canister 20 from being sucked into the vacuum housing 26 and into the vacuum source in the hospital, doctor's office, etc. Referring briefly to FIG. 7, a filter 45 may be disposed in the port 31 between the first and second ends of the port member 29.

Referring to FIGS. 4A and 4B, a set of first and second cylindrical check valves 90 are shown. Each of the check valves 90 is located in a cylindrical channel 92 of the vacuum ports 40. The check valves 90 include a valve body 90a that is fixed in place in the channel 92 and a spring biased plunger 90b that allows air to pass, i.e. permits the vacuum to be pulled within the canister 20. However, in the event that one of the vacuum ports 40 is disconnected from the vacuum source, the check valve 90 prevents air from rushing into the vacuum housing 26 from the disconnected vacuum port 40. In other words, the check valve 90 in a disconnected vacuum port 40 acts as a cap for that vacuum port 40 when not in use. This is best illustrated in FIG. 4B. The check valves 90 are preferably check valve cartridges commercially available from Neoperl, Inc. of Waterbury, Conn. An example of such a check valve is shown in U.S. Pat. No. 6,837,267 to Weis et al., herein incorporated by reference.

Referring specifically to FIG. 4B, a vacuum release button 92 is also shown. This button 92 is used to release the vacuum in the vacuum housing 26 when both the float 46 and check valves 90 have been engaged. When float 46 and valves 90 are both activated, a vacuum is created in vacuum housing 26. By depressing the button 92, the vacuum can be released and the waste collection unit 12 can be used again. The vacuum release button 92 includes a cylindrical plunger 93 disposed in a cylindrical channel 94. A spring 95 acts between the plunger 93 and a spacer 96 to bias a head of the plunger 93 into an opening in a top of the vacuum housing 26. A retainer 98 is secured in the channel 94 and supports the spacer 96. A seal 99 is disposed about a groove of the plunger 93 in the opening 97 such that when the plunger 93 is depressed, i.e., the vacuum release button 92 is activated, the seal 99 is moved and released from its seat in the opening thereby allowing air to rush into the vacuum housing 26 from the atmosphere. Gaps may be defined between the plunger 93 and the channel walls to allow the air to rush in and defeat the vacuum, or the plunger 93 may be perforated along its length for this purpose.

In use, the waste collection unit 12 is wheeled to a patient and connected to the vacuum source through the tubes 28. The manifold and filter system 32 is inserted into the inlet 22 and the collection tubes 30 are connected to the manifold and filter system 32. The vacuum tubes 28 pull a vacuum within the canister 20, which causes a vacuum to be pulled through the tubes 30 drawing in the medical waste through the collection tools. Once the procedure is completed, the vacuum tubes 28 may be disconnected and a new manifold and filter system 32, collection tubes 30 and collection tools can be connected to the waste collection unit 12 and the waste collection unit 12 can be used on another patient. Eventually, the canister 20 will become full and need to be emptied. At this point, the waste collection unit 12 is wheeled to the docking station 14 to be emptied and cleaned.

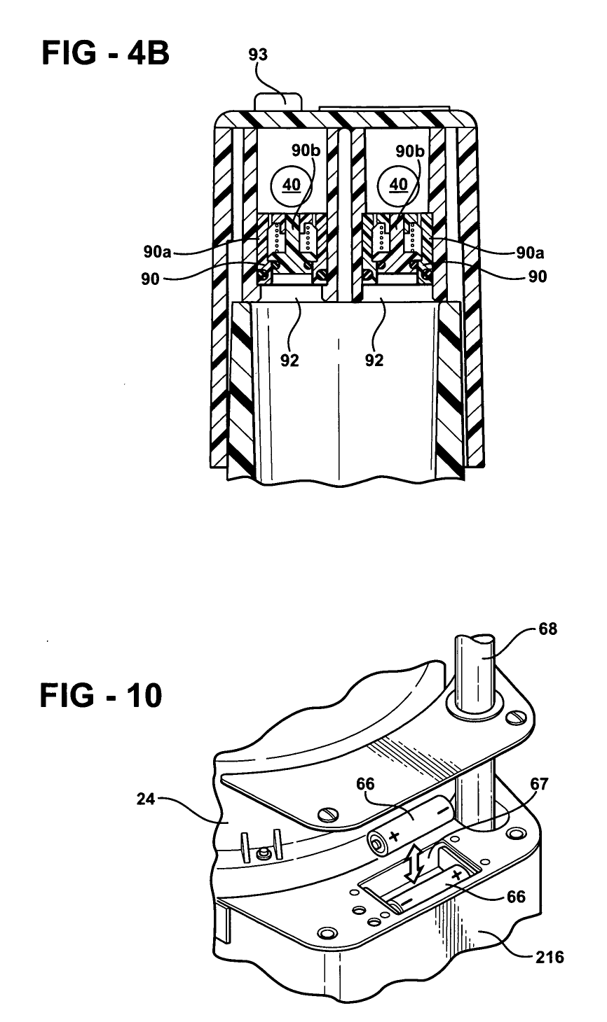

Referring to FIGS. 1 and 5A-5C, the waste collection unit 12 has a docking system 50 with a metal strike plate 80 that is floatably supported by the tower 21b. A complimentary docking receiver 52 on the docking station 14 receives the strike plate 80 to mate the waste collection unit 12 with the docking station 14. It should be appreciated that the docking system 50 could be on the docking station 14 and the receiver 52 could be on the waste collection unit 12. In the disclosed embodiment, the receiver 52 is magnetic to magnetically adhere to the strike plate 80. As should be appreciated, the plate 80 could be magnetic to adhere to a metal receiver 52 if desired. The strike plate 80, as described below, is configured to tilt relative to the tower 21b thereby offering a surface that is flat and parallel to the receiver 52, i.e., the docking station magnet, for improved magnet holding strength.

Referring specifically to FIGS. 5A-5C, a guard member 83, e.g., finger guard, is captured between the strike plate 80 and the tower 21b. The tower 21b defines a first plurality of openings 85, the guard member 83 defines a second plurality of openings 87 aligned with the first plurality of openings 85, and the strike plate 80 defines a third plurality of openings 89 aligned with the first 85 and second 87 plurality of openings. A fastener 82 is disposed in each of the aligned openings 85, 87, 89 to secure the guard member 83 between the strike plate 80 and the tower 21b. A spacer 84 is disposed about each of the fasteners 82. Each of the spacers 84 have a first diameter and each of the first 85 and second 87 plurality of openings have a second diameter greater than the first diameter to define a gap between the spacers 84 and each of the first 85 and second 87 plurality of openings. This allows movement of the spacers 84 and the fasteners 82 in all directions in each of the first 85 and second 87 plurality of openings thereby allowing the strike plate 80 to float, i.e., tilt, relative to the tower 21b. Preferably, the fasteners 82 and spacers 84 move with the strike plate 80 relative to the guard member 83 and tower 21b when the strike plate 80 is tilted.

A resilient member 86 is disposed between the guard member 83 and the strike plate 80. The resilient member 86 acts as a spring between the guard member 83 and the strike plate 80. The resilient member is further defined as a washer 86 having resilient undulations. The guard member 83 defines a pocket 83a and the washer 86 is disposed in the pocket 83a such that the resilient undulations act to bias between the strike plate 80 and the guard member 83 to floatably support the strike plate 80 relative to the guard member 83. In this way, the strike plate 80 can tilt against the bias of the washer 86 for better mating with the receiver 52. By introducing a flatter surface of the strike plate 80 to the receiver 52, which is provided by the strike plate 80 being floatably supported for tilting relative to the tower 21b, a better magnetic connection is achieved.

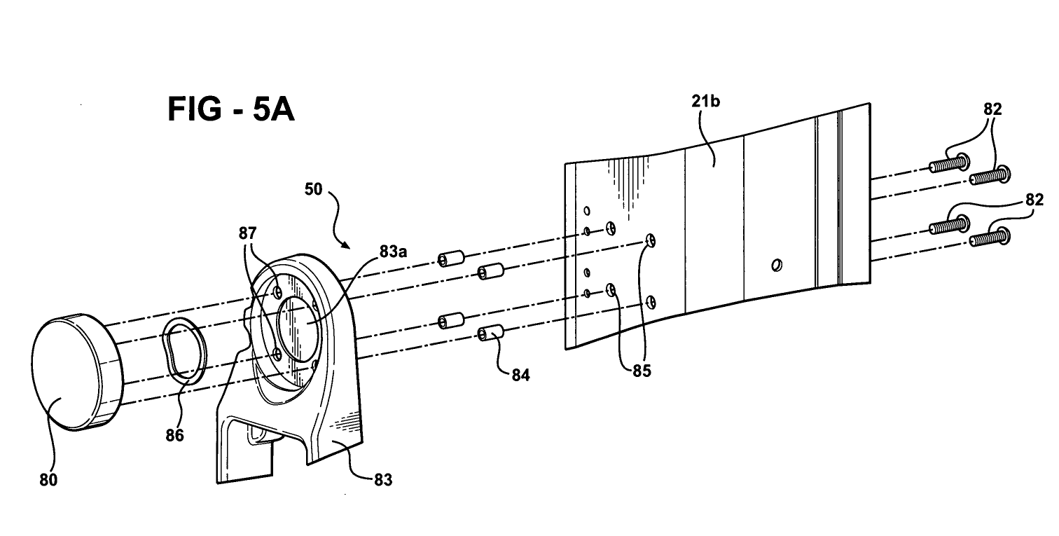

Referring to FIG. 6, the waste collection unit 12 is shown docked to the docking station 14. When this occurs, a cleaning coupling 57 is provided for receiving a cleaning agent from the docking station 14. The cleaning agent may be cleaner only, water only, a solution of cleaner and water, or any combination thereof. The cleaner is preferably an enzymatic detergent in liquid form. A waste coupling 56, which is connected to a drain 79 in the base 19 of the canister 20 by a conduit 81, is also provided for draining the waste collection unit 12 to a waste drain D at the docking station 14.

The couplings 56 and 57, which may be standard hydraulic hose couplings, are adapted to mate with complementary couplings 56a and 57a within the docking station 14. The couplings 56a and 57a are concealed within doors 58 on the docking station 14. When the waste collection unit 12 docks to the docking station 14, i.e., the strike plate 80 mates with the receiver 52, two hubs 23 of the wheels 16 simultaneously engage bumpers 25 on the docking station 14 which slide the doors 58 open (not shown). The couplings 56 and 57 on the waste collection unit 12 are then engaged by the couplings 56a and 57a from the docking station 14. More specifically, the couplings 56a and 57a from the docking station 14 are automatically moved by an actuator C, preferably a pneumatic cylinder C operated by an air compressor AC. A guide 54 on the waste collection unit 12 and a complementary pin 54a mounted to a plate 105 holding the couplings 56a, 57a aligns the couplings 56, 56a and 57, 57a to facilitate a successful fluid connection between the waste collection unit 12 and the docking station 14, as shown.

Still referring to FIG. 6, a control unit 100 of the waste collection unit 12 acts as a master control unit to a slave controller 102 of the docking station 14 to control the sequence of actuating the couplings 56a, 57a, draining the canister 20 of the medical waste, cleaning the medical waste with the cleaning agent, and further draining the cleaning agent. The control unit 100 and controller 102 communicate via corresponding IR ports 104 that align for communication purposes when the waste collection unit 12 is docked to the docking station 14.

The docking station 14 has a water line 60 extending from a water source W and a drain line 62 extending from the waste drain D. Once docked, the docking station 14 drains the canister 20 through the conduit 81, drain couplings 56, 56a, and the drain line 62 via an offloading pump P. The offloading pump P is activated once the couplings 56, 56a and 57, 57a mate, i.e., after the control unit 100 instructs the controller 102 to actuate the pneumatic cylinder C to connect the couplings 56, 56a and 57, 57a. A switch or sensor (not shown), in communication with the controller 102, may be actuated between the couplings 56, 56a or 57, 57a, or between the guide 54 and pin 54a to indicate that the couplings 56, 56a and 57, 57a have properly mated. Once empty, as detected by a float switch 106, the control unit 100 instructs the controller 102 to activate a cleaning system of the docking station 14. The cleaning system comprises a 12V solenoid valve 107 used to selectively open and close the water line 60 and an injector pump 108 used to inject a cleaner 64 into the water line 60.

The cleaning system is operated in a cleaning cycle dictated by the control unit 100, i.e., the control unit 100 (including appropriate microprocessors) is programmed to instruct the controller 102 as to the cleaning cycle, e.g., when the valve 107 should be open/closed and when cleaner 64 should be injected into the water line 60. The cleaning system transfers the cleaning agent through the cleaner couplings 57, 57a and into a sprinkler system 200 of the waste collection unit 12 shown in FIG. 7. A line 68 connects the cleaning coupling 57 to the sprinkler system 200. The sprinkler system 200 directs the cleaning agent inside of the canister 20 during the cleaning cycle to clean the canister 20. During normal operation of the cleaning cycle, the cleaning agent includes a ratio of cleaner to water of from 1:80 to 1:130, most preferably 1:128 or 1 ounce of cleaner per gallon of water.

Referring to FIG. 7, the sprinkler system 200 is shown. The sprinkler system 200 includes a spiral cleaning nozzle 70 or sprinkler 70. This sprinkler 70 includes a spiral or helical configuration that provides a radial spray array in the form of a cone-shaped spray pattern, as illustrated, to impact all interior surfaces of the wall 27 of the canister 20 with the cleaning agent to clean the canister 20. Other sprinklers can be used, but the sprinkler 70 provides an advantageous spray pattern that facilitates cleaning of the canister 20. The cap 24 supports the sprinkler 70. A nozzle 71 is fixed in a central opening in the cap 24. The nozzle 71 connects to the line 68 at a first end outside of the canister 20 and extends into the canister 20 to a second end. A coupling 73 interconnects the nozzle 71 and the sprinkler 70 inside the canister 20. The nozzle 71 may be fixed in the cap 24 by adhesive, a threaded connection, or other conventional methods. The coupling 73 may be fixed to the nozzle 71 and the sprinkler 70 in a similar fashion. A check valve 75, similar to those used in the vacuum ports 40 may be provided in the nozzle 71 to prevent the medical waste from exiting out through the nozzle 71 when the waste collection unit 12 is in use. A perspective view of the sprinkler 70 is shown in FIG. 3.

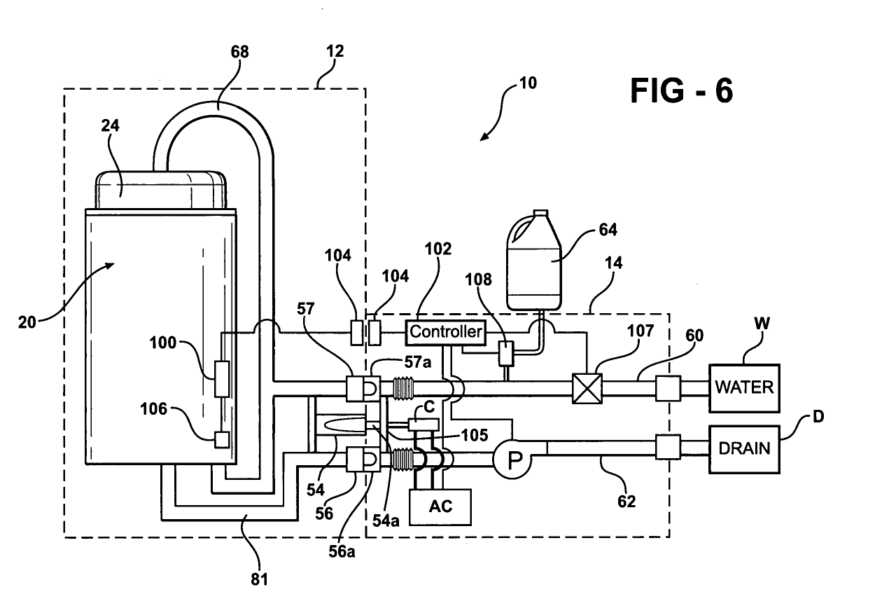

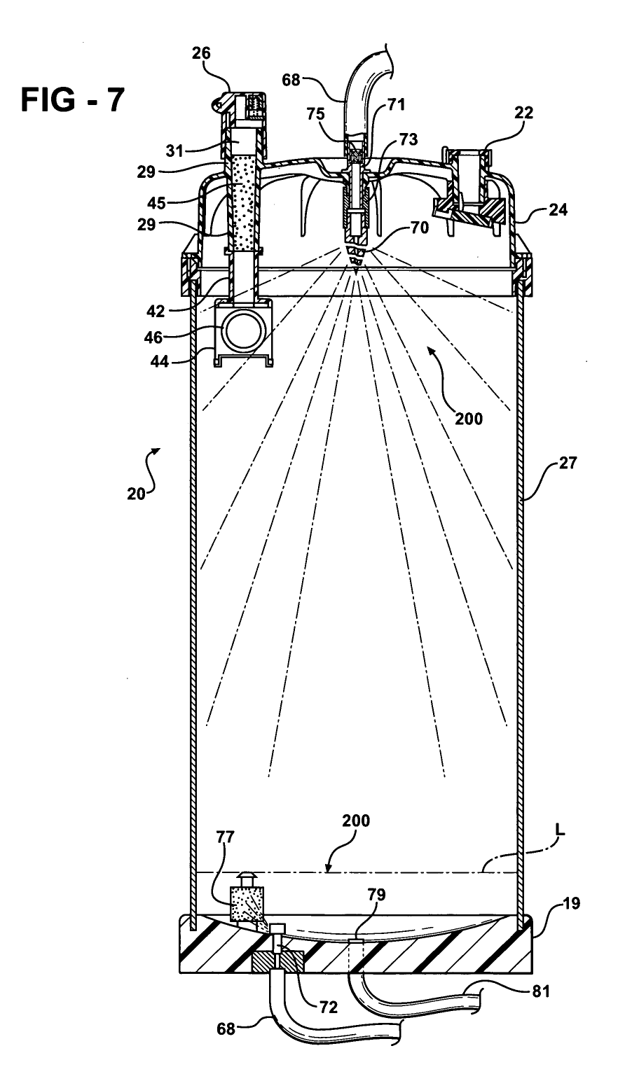

The sprinkler system 200 also includes a spray nozzle 72 disposed in the base 19. The spray nozzle 72 is adjacent to a float 77 slidably mounted in an opening 74 (see FIGS. 8 and 9) in the base 19 of the canister 20. The float 77 actuates the float switch 106 to electronically transmit a signal to the control unit 100 and subsequently to the controller 102 of the docking station 14 that the canister 20 is empty. The spray nozzle 72 sprays the cleaning agent directly at the float 77 to remove debris from the float 77 and ensure that the float 77 does not become clogged and inoperative. One end of the spray nozzle is connected to the line 68 to receive the cleaning agent.

The float 77 can be totally submerged during cleaning if desired. In this instance, the control unit 100 is programmed for filling the canister 20 with the cleaning agent to a level L above the float 77 and draining the cleaning agent from the canister 20 after the float 77 is submersed such that the float 77 is further cleaned. The control unit 100 utilizes the offload pump P and cleaning system of the docking station 14 via the slave controller 102 to perform these functions. A drain hole, or outlet, for the canister 20 is shown at 79 and is in communication with drain coupling 56. The direction of flow from the spray nozzle 72 is circular along the base 19 of the canister 20. This circular flow breaks up sediment in a bottom 75 of the canister 20 to facilitate cleaning when disposing of the medical waste at the docking station 14. This is best shown in FIGS. 8 and 9.

Referring back to FIGS. 6 and 7, the control unit 100 is programmed for supplying the cleaning agent at a high concentration into the canister 20 through the sprinkler system 200 in a plurality of repeated injections during the cleaning cycle with pauses between the injections. To perform this function, the control unit 100 instructs the controller 102 to load the cleaner into the water line 60 via injector pump 108 without opening the valve 107. This loads the water line 60 with cleaner. Then the controller 102 is instructed to repeatedly open and close the valve 107 such that the cleaner is injected through the sprinkler system 200 in the plurality of repeated injections. This results in the cleaning agent having a high concentration of cleaner with little water to facilitate the injections. Here, the cleaning agent includes a volumetric ratio of cleaner to water of from 1:1 to 1:30, most preferably 1:10.

Between injections, the cleaning agent acts against the wall 27, the float 77, and the base 19 and is allowed to work for a few seconds. The injections may be repeated six or seven times. The cleaning cycle may end with a full rinse of water. The cleaning agent, including cleaner and water, is then drained from the canister 20 via the offload pump P resulting in the waste collection unit 12 being clean and ready for further use.

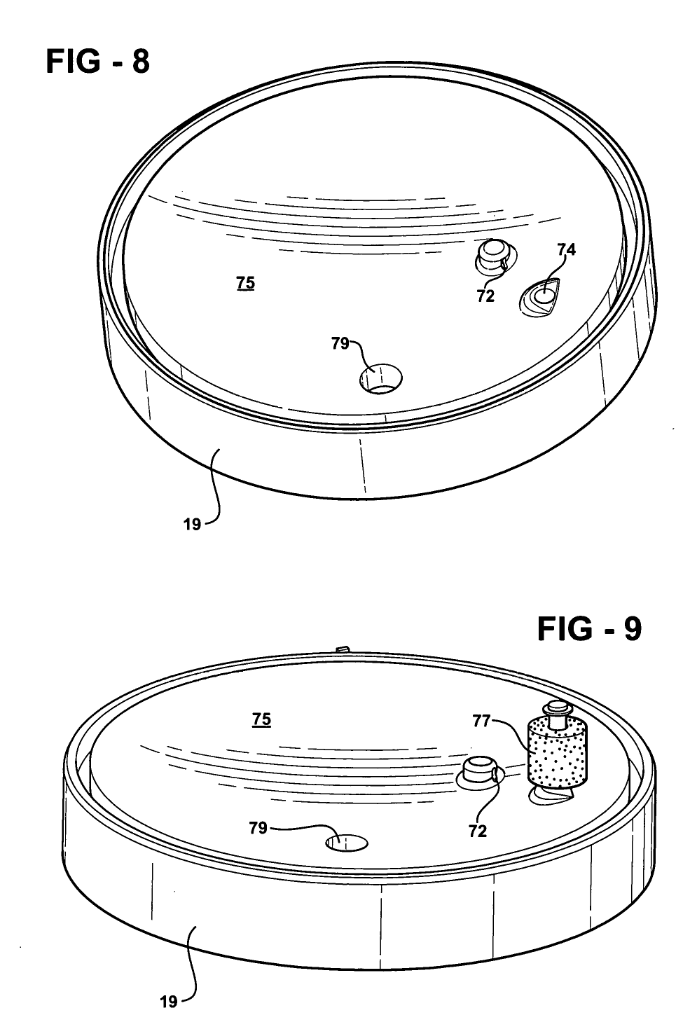

Referring to FIG. 10, the control unit 100 is powered by batteries 66 to communicate with the docking station 14 and control the sequence of draining, cleaning, and further draining. In the preferred embodiment, the batteries 66 are two double AA batteries and a battery compartment 67 is defined in the tower 21b of the carrier 21 to house the batteries 66. The control unit 100 also has a sleep mode to increase the useable life of the batteries 66. In one embodiment, the docking station 14 has a battery charger (not shown) to recharge the batteries 66 on the waste collection unit 12. The portable unit 12 can also be powered and recharged through the docking receiver 52. As should be appreciated, the control unit 100 and power supply could be housed in the docking station 14 if desired.

Obviously many modifications and variations of the present invention are possible in light of the above description. While this description is directed to particular embodiments, it is understood that those skilled in the art may conceive of modifications and/or variations to the specific embodiments shown and described herein. Any such modifications or variations, which fall within the purview of this description, are intended to be included herein as well. It is understood that the description herein is intended to be illustrative only and is not intended to be limited. Rather, the scope of the invention described herein is limited only by the claims appended hereto.

1. A waste collection unit for collecting medical waste, comprising:
a canister having an inlet for receiving the medical waste and an outlet for discharging the medical waste, said canister including a cap and a base with at least one wall extending between said cap and said base; and
a sprinkler supported by said cap for directing a cleaning agent against said wall of said canister during a cleaning cycle wherein said sprinkler includes a configuration for providing a radial spray array of the cleaning agent against said wall of said canister.

2. A waste collection unit as set forth in claim 1 wherein said configuration is further defined as a helical configuration for providing a cone-shaped spray pattern of the cleaning agent against said wall of said canister.

3. A waste collection unit as set forth in claim 1 including at least one spray nozzle disposed in said base for cleaning said base and said wall of said canister.

4. A waste collection unit as set forth in claim 3 including a float disposed in said base of said canister.

5. A waste collection unit as set forth in claim 4 wherein said at least one spray nozzle is disposed in said base adjacent to said float and said at least one spray nozzle is directed at said float such that the cleaning agent can be sprayed through said at least one spray nozzle onto said float during said cleaning cycle to remove debris from said float.

6. A waste collection unit as set forth in claim 3 including a control unit programmed for controlling a supply of the cleaning agent to said sprinkler and said spray nozzle.

7. A waste collection unit as set forth in claim 6 wherein said control unit is programmed for supplying the cleaning agent at a high concentration into said canister through said sprinkler and said at least one spray nozzle in a plurality of repeated injections during said cleaning cycle with pauses between said injections.

8. A waste collection unit as set forth in claim 6 wherein said control unit is programmed for filling said canister with the cleaning agent to a predetermined level and draining the cleaning agent from said canister to further clean said canister.

9. A waste collection unit as set forth in claim 6 wherein said control unit includes an infrared port for communicating with a controller of a docking station to control the supply of the cleaning agent to said sprinkler and said at least one spray nozzle during said cleaning cycle.

10. A waste collection unit as set forth in claim 6 including a carrier supporting said canister with a battery compartment defined in said carrier for housing batteries used to power said control unit wherein said control unit includes a sleep mode for increasing the useable life of the batteries.

11. A waste collection unit as set forth in claim 10 wherein said carrier includes a plurality of wheels for providing mobility to said waste collection unit.

12. A waste collection unit as set forth in claim 11 wherein said carrier includes a handle for maneuvering said waste collection unit between use areas.

13. A waste collection unit for collecting medical waste, comprising:
a canister having an inlet for receiving the medical waste and an outlet for discharging the medical waste, said canister including a cap and a base with at least one wall extending between said cap and said base;
a sprinkler system for directing a cleaning agent inside of said canister during a cleaning cycle to clean said canister; and
a control unit programmed for supplying the cleaning agent at a high concentration into said canister through said sprinkler system in a plurality of repeated injections during said cleaning cycle with pauses between said injections.

14. A waste collection unit as set forth in claim 13 wherein said sprinkler system includes a sprinkler supported by said cap wherein said sprinkler includes a configuration for providing a radial spray array of the cleaning agent to clean said canister during said cleaning cycle.

15. A waste collection unit as set forth in claim 13 wherein said sprinkler system includes at least one spray nozzle disposed in said base for cleaning said base and said wall of said canister.

16. A waste collection unit as set forth in claim 15 including a float disposed in said base of said canister.

17. A waste collection unit as set forth in claim 16 wherein said at least one spray nozzle is disposed in said base adjacent to said float and said at least one spray nozzle is directed at said float such that the cleaning agent can be sprayed through said at least one spray nozzle onto said float during said cleaning cycle to remove debris from said float.

18. A waste collection unit as set forth in claim 13 wherein said control unit is programmed for filling said canister with the cleaning agent to a predetermined level and draining the cleaning agent from said canister to further clean said canister.

19. A waste collection unit as set forth in claim 13 wherein said control unit includes an infrared port for communicating with a controller of a docking station to operate a cleaning system of the docking station during said cleaning cycle.

20. A waste collection unit as set forth in claim 13 including a carrier supporting said canister with a battery compartment defined in said carrier for housing batteries used to power said control unit wherein said control unit includes a sleep mode for increasing battery life.

21. A waste collection unit as set forth in claim 20 wherein said carrier includes a plurality of wheels for providing mobility to said waste collection unit.

22. A waste collection unit as set forth in claim 21 wherein said carrier includes a handle for maneuvering said waste collection unit between use areas.

23. A waste collection unit for collecting medical waste, comprising:
a canister having an inlet for receiving the medical waste and an outlet for discharging the medical waste;
a vacuum housing having first and second vacuum ports in communication with said canister for selectively connecting to an external vacuum source to draw a vacuum within said canister thereby drawing the medical waste into said canister through said inlet; and
a check valve disposed within said first vacuum port for preventing the drawing of air in through said first vacuum port while said second vacuum port is connected to the external vacuum source and said first vacuum port is disconnected from the external vacuum source.

24. A waste collection unit as set forth in claim 23 including a second check valve disposed within said second vacuum port for preventing the drawing of air in through said second vacuum port while said first vacuum port is connected to the external vacuum source and said second vacuum port is disconnected from the external vacuum source.

25. A waste collection unit as set forth in claim 24 wherein said canister includes a cap with a port defined in said cap and said vacuum housing is mounted about said port to provide said communication between said vacuum ports and said canister.

26. A waste collection unit as set forth in claim 25 wherein said cap includes an integrally formed port member further defining said port and having a first end outside of said canister and a second end inside of said canister.

27. A waste collection unit as set forth in claim 26 including a cage mounted to said second end of said port member with a check ball disposed within said cage whereby said check ball is lifted by the medical waste contained in said canister when said medical waste rises to a predetermined level such that said check ball closes said communication between said vacuum ports and said canister when the medical waste is at the predetermined level.

28. A waste collection unit as set forth in claim 27 including a vacuum release button supported by said vacuum housing for releasing a vacuum inside of said vacuum housing.

29. A waste collection unit as set forth in claim 28 wherein said vacuum housing defines an opening in a top of said vacuum housing and said vacuum release button comprises a plunger protruding into said opening.

30. A waste collection unit as set forth in claim 29 wherein said vacuum release button further comprises a spring for biasing said plunger into said opening and a seal for sealing about said plunger within said opening such that when said vacuum release button is depressed said seal is moved and released and the vacuum inside of said vacuum housing is exposed to atmosphere.

31. A waste collection unit as set forth in claim 26 wherein said vacuum housing is mounted to said port member at said first end.

32. A waste collection unit as set forth in claim 26 including a filter disposed in said port between said first and second ends of said port member.

33. A waste collection unit as set forth in claim 23 including a carrier supporting said canister wherein said carrier includes a plurality of wheels for providing mobility to said waste collection unit.

34. A waste collection unit as set forth in claim 33 wherein said carrier includes a handle for maneuvering said waste collection unit between use areas.

35. A waste collection unit for collecting medical waste and disposing of the medical waste at a docking station, comprising:
a canister having an inlet for receiving the medical waste and an outlet for discharging the medical waste at the docking station;
a carrier supporting said canister and including a plurality of wheels for providing mobility to said waste collection unit between use areas and the docking station; and
a strike plate floatably supported by said carrier for mating with the docking station.

36. A waste collection unit as set forth in claim 35 wherein said strike plate tilts relative to said carrier thereby offering a surface that is flat and parallel to a docking station magnet for improved magnet holding strength.

37. A waste collection unit as set forth in claim 36 including a guard member captured between said strike plate and said carrier.

38. A waste collection unit as set forth in claim 37 wherein said carrier defines a first plurality of openings, said guard member defines a second plurality of openings aligned with said first plurality of openings, and said strike plate defines a third plurality of openings aligned with said first and second plurality of openings.

39. A waste collection unit as set forth in claim 38 including a fastener disposed in each of said aligned openings to secure said guard member between said strike plate and said carrier.

40. A waste collection unit as set forth in claim 39 including a spacer disposed about each of said fasteners.

41. A waste collection unit as set forth in claim 40 wherein each of said spacers have a first diameter and each of said first and second plurality of openings have a second diameter greater than said first diameter to define a gap between said spacers and each of said first and second plurality of openings to allow movement of said spacers and said fasteners in each of said first and second plurality of openings thereby allowing said strike plate to tilt relative to said carrier.

42. A waste collection unit as set forth in claim 38 including a resilient member disposed between said guard member and said strike plate.

43. A waste collection unit as set forth in claim 42 wherein said resilient member is further defined as a washer having resilient undulations.

44. A waste collection unit as set forth in claim 43 wherein said guard member defines a pocket and said washer is disposed in said pocket such that said resilient undulations act between said strike plate and said guard member to floatably support said strike plate relative to said guard member.

45. A waste collection unit as set forth in claim 35 wherein said carrier includes a handle for maneuvering said waste collection unit between the use areas and the docking station.

46. A waste collection unit for collecting medical waste and docking with a docking station to discharge the medical waste to a drain at the docking station, comprising:
a canister having an inlet for receiving the medical waste and an outlet for discharging the medical waste at the docking station;
a vacuum housing having at least one vacuum port in communication with said canister for selectively connecting to an external vacuum source to draw a vacuum within said canister thereby drawing the medical waste into said canister through said inlet; and
a control unit for automatically providing fluid communication between said outlet and the drain at the docking station when said waste collection unit is docked to the docking station thereby allowing discharge of the medical waste from said canister without requiring any manual connections between said waste collection unit and the docking station.

47. A waste collection unit as set forth in claim 44 wherein said control unit is programmed for instructing the docking station to operate an offload pump of the docking station during an offload cycle to drain the medical waste from said canister.

48. A waste collection unit as set forth in claim 45 wherein said control unit is programmed for instructing the docking station to operate a cleaning system of the docking station during a cleaning cycle to clean said canister.

49. A waste collection system as set forth in claim 46 including a sprinkler system for directing a cleaning agent inside of said canister during the cleaning cycle to clean said canister.

50. A waste collection unit as set forth in claim 47 wherein said control unit includes an infrared port for communicating with a controller of the docking station to control a supply of the cleaning agent to said sprinkler system.

51. A waste collection unit as set forth in claim 44 including a carrier supporting said canister with a battery compartment defined in said carrier for housing batteries used to power said control unit wherein said control unit includes a sleep mode for increasing the useable life of the batteries.

52. A waste collection unit as set forth in claim 49 wherein said carrier includes a plurality of wheels for providing mobility to said waste collection unit.

53. A waste collection unit as set forth in claim 50 wherein said carrier includes a handle for maneuvering said waste collection unit between use areas and the docking station.

54. A method of collecting and disposing of medical waste using a waste collection unit having a canister for containing the medical waste wherein the waste collection unit is adapted for docking with a docking station to discharge the medical waste from an outlet of the canister to a drain at the docking station, said method comprising the steps of:
connecting the waste collection unit to an external vacuum source to draw a vacuum within the canister and collect medical waste from at least one surgical procedure;
disconnecting the waste collection unit from the external vacuum source after the canister is at least partially filled with the medical waste;
docking the waste collection unit to the docking station after disconnecting the waste collection unit from the external vacuum source and after the waste collection unit is at least partially filled with the medical waste; and
automatically providing fluid communication between the outlet of the canister and the drain at the docking station when the waste collection unit is docked to the docking station without requiring any manual connections between the waste collection unit and the docking station.

55. A method as set forth in claim 54 including automatically discharging the medical waste from the canister once fluid communication is provided between the outlet and the drain.

56. A method as set forth in claim 55 including sensing an empty condition of the canister after discharging the medical waste from the canister.

57. A method as set forth in claim 56 including automatically cleaning the canister after sensing the empty condition.
